(19) United States
(12) Patent Application Publication
(10) Pub. No.: US 2005/0267382 A1
(43) Pub. Date: Dec. 1, 2005

(54) Sensor

(76) Inventors: Jonathan Mark Church, Auckland (NZ); Warrick Heald, Auckland (NZ); Neil Prime, Auckland (NZ)

Correspondence Address: TREXLER, BUSHNELL, GIANGIORGI,;BLACKSTONE & MARR, LTD., 105 WEST ADAMS STREET, SUITE 3600, CHICAGO, IL, 60603, US

(21) Appl. No.: 11/122,166

(22) Filed: May 4, 2005

(30) Foreign Application Priority Data
May 4, 2004 (NZ) 532729

(51) Int. Cl. A61G 11/00; G01K 1/00; G01K 13/12; A61B 5/00

(52) U.S. Cl. 600/549; 374/100

(57) ABSTRACT

The present invention relates to a Hydrogel cover for a temperature sensor. The sensor can be used on infants in a radiant warmer and/or incubator. In a first embodiment the sensor is fully enclosed by the Hydrogel. In a second embodiment the sensor may be removed through a releasable cover. This results in improved thermal response and/or accuracy.

### FIELD OF THE INVENTION

The present invention relates to a skill temperature sensor particularly though not solely for use with infant radiant warmers or convective incubator.

### BACKGROUND

In the U.S. Pat. No. 5,841,944, the content of which is incorporated by reference, a radiant warmer is described for heating an infant. As an integral part of the warmer a temperature sensor is required to measure the infants skin temperature at all times, to allow the radiant heater closed loop control. The sensor must maintain contact with the infant's skin and reflect away radiant energy from the heater above.

Traditionally there has been a separate temperature sensor and a separate cover where the sensor is attached to the patient with the cover placed on top of it. There are also difficulties with the cover providing adequate pressure on the sensor and removing the cover from the infants' skin without damage.

An example of the prior art is a stainless steel disc containing two temperature sensing thermistors. They can be used in conjunction with a separate disposable probe cover to adhere the probe to the skin surface and insulate the probe from the radiant heat from the warmer to ensure an accurate skin temperature measurement In FIG. 5 the foil 501 reflects infa-red heat from a radiant warmer, the insulating foam 502 acts as an insulation layer for the temperature sensor. The sensor rests on the skin and the Hydrogel 503 (which is skin adhesive with the backing plastic 504 removed just prior to application) adheres over the sensor onto the surrounding skin

### SUMMARY OF THE INVENTION

It is therefore an object of the present invention to over any disadvantages in the prior ant or which will at least provide the public with a useful choice.

Accordingly in a first aspect the present invention consists in a system for determining the temperature of a patient's skin comprising:
an adhesive polymer matrix configured to adhere to a portion of said patient's skin,
a receptacle for a temperature sensor at least part thermally contacting said polymer matrix, and
a thermally reflective or shielding panel on the opposite side of said receptacle to that which would in use be proximate the skin of said patient.

Preferably said receptacle is enclosed by said panel, enclosed by said polymer matrix or enclosed by a combination of said panel and said polymer matrix.

Preferably further comprising a temperate sensor within said receptacle.

Preferably said patient is an infant.

Preferably said polymer matrix in use adhered to said panel.

Preferably said sensor including an electrical connection and said polymer matrix in use adhered to said connection.

Preferably said polymer matrix in use adhered to said sensor.

Preferably said panel further comprising a radiant shield, and thermal insulation between said shield and said sensor.

Preferably said polymer matrix is a polymerizerable polymer Hydrogel.

Preferably said sensor is fully encapsulated by said polymer matrix.

Preferably said polymer matrix includes a detachable lid to encapsulate said sensor.

Preferably said lid includes a loop fastening portion.

Preferably said lid is comprised of a further portion of said polymer matrix which adheres to the main portion thereby encapsulating said sensor.

Preferably said sensor is a flat disc type sensor.

Preferably said shield is mylar foil.

Preferably said mylar foil is 0.025 mm thick.

Preferably said insulation is foam.

Preferably said foam is similar or the same in thickness to said sensor or said receptacle.

To those skilled in the art to which the invention relates, many changes in construction and widely differing embodiments and applications of the invention will suggest themselves without departing from the scope of the invention as defined in the appended claims. The disclosures and the descriptions herein are purely illustrative and are not intended to be in any sense limiting.

The invention consists in the foregoing and also envisages constrictions of which the following gives examples.

### BRIEF DESCRIPTION OF THE DRAWINGS

Examples of the present invention will now be described with reference to the accompanying drawings in which;

FIG. 1 is a blown out perspective of the first embodiment of the present invention.

FIG. 2 is a blown out perspective of the second embodiment of the present invention.

FIG. 3 is a blown out perspective of the third embodiment of the present invention,

FIG. 4 is a perspective of the present invention in use, and

FIG. 5 is a prior art temperature sensor adhesive cover.

### DETAILED DESCRIPTION

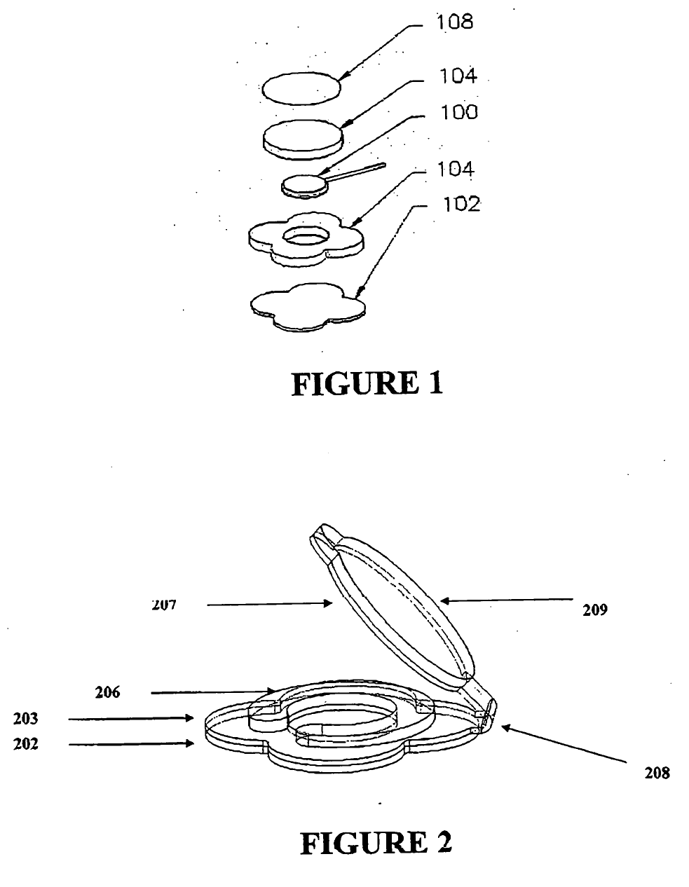

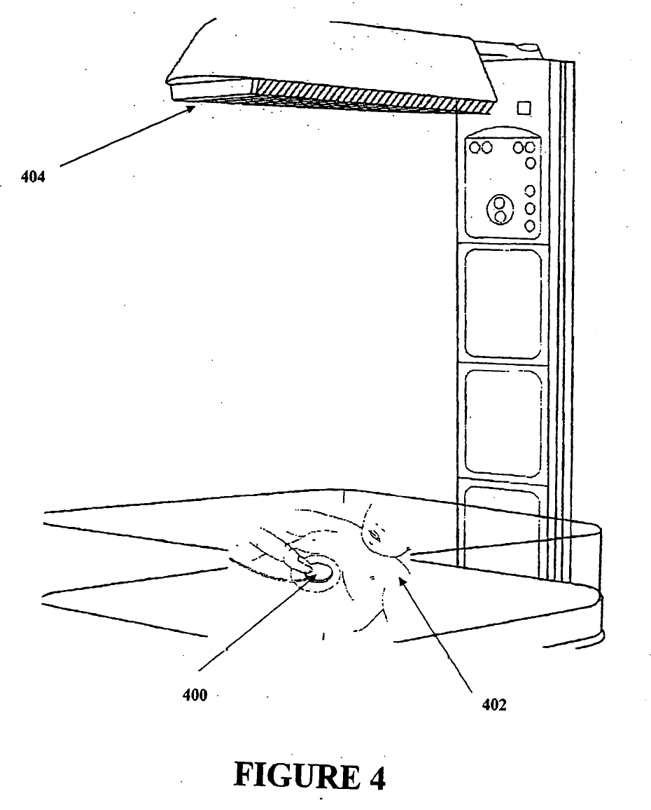

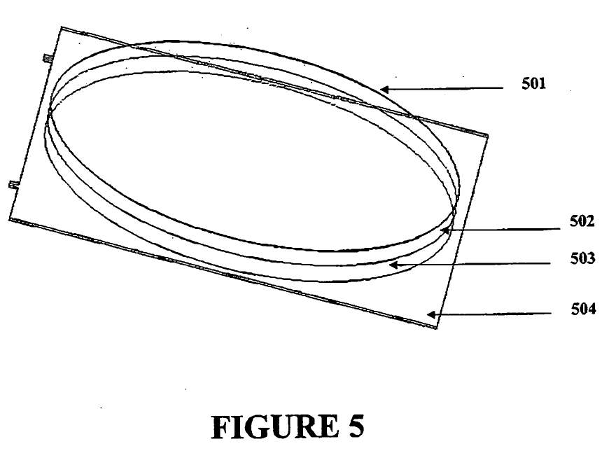

In a first embodiment the present invention comprises an all in one temperature sensor and cover. The sensor 100 would be sandwiched between a bottom layer of Hydrogel 102 that adheres to the patient's skin, and a top thermal shield layer of foam 104 and reflective metallised polyester 108 seen in FIG. 1. The sensor pad 400 would be attached to the infant 402 on an infant warmer 404 as seen in FIG. 4. This can be used in radiant warmers such as disclosed in U.S. Pat. No. 5,841,944, the contents of which are incorporated herein by reference, and/or infant incubators, although other applications will be apparent to one skilled in the art.

Measuring the patient's temperature through the Hydrogel a more accurate result is gained than with prior art infant sensing. The sensor is totally enclosed eliminating temperature fluctuations due to air gaps and the sensor is more secure and less likely to pull away from the patient. The Hydrogel used is such that the adhesiveness of it will hold the sensor in place but won't remove infant's skin when being removed.

The thermal conductivity of the Hydrogel is such that any change in skin temperature is transferred to the sensor as quickly and with less noise than without the Hydrogel. The present invention may also comprise a sensor enclosure for a conventional temperature sensor. There are two approaches here.

1) In a second embodiment referring to FIG. 2 the temperature sensor 100 is preferably placed on top of the Hydrogel 202 and foam 203 and then enclosed by a Velcro fastening arrangement 206, 207. The sensor is preferably held in place by the tackiness of the Hydrogel 202 and the Velcro fling arrangement 204. The fastening arrangement 204 preferably has a metallised polyester reflective panel 206 on top of it to reflect the radiant heat away. The Velcro 204 allows the top panel 208 to lift and the sensor 200 to be removed easily. The hook fastener would be a limited use fastener.

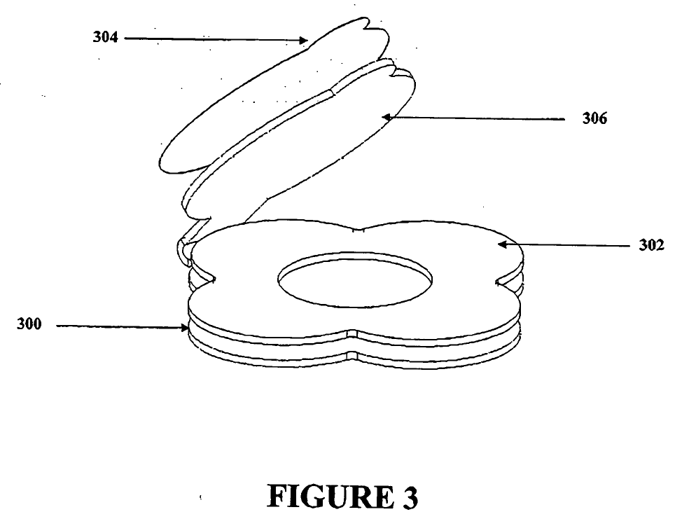

2) In a third embodiment referring to FIG. 3 similar to the above, with the Velcro being replaced by an all in one Hydrogel 300 and foam 302 arrangement. The sensor sits on top of the Hydrogel 300 surrounded by foam 302 and is held in by the bottom Hydrogel's tackiness along with another layer of Hydrogel 306 folded on top of it covered with a reflective metallised polyester layer 304. The sensor can be removed by pulling back the top layer 306 of Hydrogel.

Each portion of Hydrogel and/or the other components would be die cut and then manually assembled. The assemblies are placed on a release liner and packaged in foil barrier pouches.

Hydrogel

The Hydrogel is required to be adhesive to the skin of mammals, particularly that of infants, without causing damage on removal. Examples are given in U.S. Pat. No. 5,868,136, U.S. Pat. No. 6,038,464 and U.S. Pat. No. 6,115,625, the contents of which incorporated herein by reference.

Preferably the Hydrogels is chosen from the range currently offered by Amgel Corporation. The Amgel Hydrogels are characterised by an internal screen which allows two different adhesion properties in one gel. Thus the Hydrogel can be formulated to have optimum skin adhesion to the patient on one surface and strong adhesion to substrates to fabricate the cover itself. AG 603 type Hydrogel is at the high end of the skin adhesion range of neonates. AG 603 appears to have the best properties of the Hydro gel tested

The Sensor

A stainless steel disc, PVC tip, Mylar tip and the over moulded with plastic are all possible constructions of the sensor.

The PVC tip cap did not perform well. It is difficult to get good contact with the block and generally there is always an air gap, which acts as an insulator causing the temperature sensor to under read by up to 1° C. The application of a thermally conductive potting compound allowed the tip cap sensor to read accurately confirming the air gap theory. The stainless steel disc samples read accurately throughout the tests.

For a flat disc type sensor sensing through the Hydrogel shows comparable accuracy and response to the conventional method. This method is likely to have the advantages of maximum adhesion from full contact area and reducing the propensity to have air gaps.

The Thermal Shield

The Mylar foil and the foam both have the effect of cutting down the radiated heat from the warmer contributing to the skin temperature measurement. Prior art products which do not have foam have an artificially high skin temperature reading and undergo transients when the applied heat is suddenly removed i.e. putting an absorber over the block. The sensor through the Hydrogel is at least sensitive and reflects the block temperature more accurately than with the prior art. The Mylar foil preferably covers the whole area of the Hydrogel, as this is the most efficient at resisting the effects of radiant heat. Preferably the foam is the same thickness as the sensor (in this case 2.4 mm) and the foil is 0.025 mm thick.

To have as accurate temperature measurement as possible a full foam and Mylar covering is preferred. Prior art probe covers give artificially high temperature readout because they do not have a layer of foam insulation.

The removal of air from the system is preferable. Variations in temperature measurement have generally been associated with entrapment of air either around the sensor itself or between layers in the probe covers construction.

The first embodiment may be particularly suited for Labour and Delivery applications in that the use of the sensor would be in the order of hours with maximum feedback required in the first 15-20 minutes. This is the time taken to stabilize the baby and prepare it for transport to the Neonatal Intensive Care Unit (NICU) where the infants are cared for on a long term basis.

For the NICU itself this design would work well if the Hydrogel had longevity of up to 72 hours, but due to the possible length of stay of a neonate in the NICU it may probably more advantageous to use the second or third embodiment with a separate probe cover and temperature sensor. The Hydrogel is only effective for a certain period and therefore ideally should be replaced without the need to replace or reconnect the sensor.

1. A system for determining the temperature of a patieint's skin comprising:
an adhesive polymer matrix configured to adhere to a portion of said patient's skin,
a receptacle for a temperature sensor at least part thermally contacting said polymer matrix, and
a thermally reflective or shielding panel on the opposite side of said receptacle to that which would in use be proximate the skin of said patient.

2. A system as claimed in claim 1 wherein said receptacle is enclosed by said panel, enclosed by said polymer matrix or enclosed by a combination of said panel and said polymer matrix.

3. A system as claimed in claim 1 flier comprising a temperature sensor within said receptacle.

4. A system as claimed in claim 2 wherein said patient is an infant.

5. A system as claimed in claim 2 wherein said polymer matrix in use adhered to said panel.

6. A system as claimed in claim 2 wherein said sensor including an electrical connection and said polymer matrix in use adhered to said connection.

7. A system as claimed in claim 2 wherein said polymer matrix in use adhered to said sensor.

8. A system as claimed in claim 3 wherein said panel further comprising a radiant shield, and thermal insulation between said shield and said sensor.

9. A system as claimed in claim 7 wherein said polymer matrix is a polymerizerable polymer Hydrogel.

10. A system as claimed in claim 2 wherein said sensor is fully encapsulated by said polymer matrix.

11. A system as claimed in claim 2 wherein said polymer matrix includes a detachable lid to encapsulate said sensor.

12. A system as claimed in claim 10 wherein said lid includes a loop fastening portion.

13. A system as claimed in claim 10 wherein said lid is comprised of a further portion of said polymer matrix which adheres to the main portion thereby encapsulating said sensor.

14. A system as claimed in claim 2 wherein said sensor is a flat disc type sensor.

15. A system as claimed in claim 7 wherein said shield is mylar foil.

16. A system as claimed in claim 14 wherein said mylar foil is 0.025 mm thick.

17. A system as claimed in claim 15 wherein said insulation is foam.

18. A system as claimed in claim 16 wherein said foam is similar or the same in thickness to said sensor or laid receptacle.
